SEQUENCE LISTING (2 sequences)

SEQ ID NO: 1 - DNA, 33 bp, homo sapiens
ttgaattcataatggtagagttcgcgcccttgt

SEQ ID NO: 2 - DNA, 26 bp, homo sapiens
accggtgcagaactccaagtgctggt

(19) United States
(12) Patent Application Publication
(10) Pub. No.: US 2011/0275647 A1
(43) Pub. Date: Nov. 10, 2011

(54) PYRIMIDIN-4-(3H)-ONE DERIVATIVES

(75) Inventors: Keisuke Arakawa, Ibaraki (JP); Yoshiki Ito, Ibaraki (JP); Hiroshi Kawamoto, Ibaraki (JP); Junichi Sakaki, Shizuaka (JP); Tadashi Shimamura, Ibaraki (JP)

(73) Assignee: MSD K.K., Chiyoda-ku, Tokyo (JP)

(21) Appl. No.: 13/144,316

(22) PCT Filed: Feb. 22, 2010
(86) PCT No.: PCT/JP2010/053179

§ 371 Date: Jul. 13, 2011

(30) Foreign Application Priority Data
Feb. 23, 2009 (JP) 2009-039497

(51) Int. Cl. A61K 31/501; A61K 31/519; A61P 3/06; A61P 3/10; A61P 3/04; A61K 31/497; C07D 471/04

(52) U.S. Cl. 514/252.02; 514/255.05; 514/264.1; 544/238; 544/279

(57) ABSTRACT

The present invention relates to a compound represented by a formula (I): or a pharmaceutically acceptable salt thereof, wherein R¹ represents lower alkyl or the like; R² represents phenyl or the like; R represents a halogen atom or the like; X represents an oxygen atom or the like; Y₁, Y₂, Y₃ and Y₄ represent CH or the like; l represents an integer of from 0 to 3; m and n each represent an integer of 1 or 2; p represents an integer of from 0 to 2; and q represents an integer of from 1 to 3.

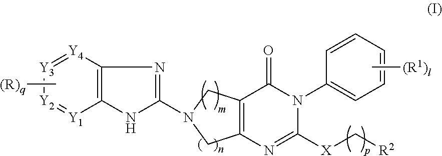

### TECHNICAL FIELD

The present invention relates to pyrimidin-4(3H)-one derivatives which are useful in the pharmaceutical field. These compounds have inhibitory activity of monoacylglycerol acyltransferase type 2 (hereinafter also referred to as “MGAT2”) and are useful as agents for treating and/or preventing hyperlipidemia, diabetes and obesity.

### BACKGROUND ART

Obesity is a condition, in which the background of lack of exercise, intake of excessive energy, ageing, etc. leads to an energy imbalance in the body, the surplus energy is accumulated generally as neutral fat (triacylglycerol, TG) in adipose tissue, and thus body weight and fat mass are increased. In recent years, the concept of metabolic syndrome associated with obesity involving the accumulation of the visceral fat, as an upstream risk factor including a plurality of risk factors of diabetes, lipidosis, hypertension, etc. has been established, and the diagnostic criteria and therapeutic guidelines for the metabolic syndrome were formulated (Journal of Japan Society for the Study of Obesity, vol. 12, Extra Edition, 2006). Since the metabolic syndrome results in increase in the risks of arteriosclerosis, cardiovascular disorder and cerebrovascular disorder, treatment of obesity has been recognized to be important for preventing these diseases.

Although the need of treating obesity is recognized to be important, there are extremely-limited drug therapies for obesity that are currently available, and there is no drug that is sufficiently satisfactory in terms of drug efficacy or side effects. Thus, development of novel antiobestic drugs having more definite action and few side-effects is desired.

Not less than 90% of lipid present in food is TG. TG derived from food is decomposed into 2-monoacylglycerol (2-MG) and free fatty acid (FFA) by the cleavage of the ester linkages of aliphatic acids at the 1- and 3-positions by lipase in digestive juice, which is secreted from the pancreas and the stomach. The 2-MG and FFA as well as bile acid are micellized and absorbed into small intestinal epithelial cells. The absorbed 2-MG and FFA resynthesize TG in the small intestinal cells, and the resynthesized TG as lipoprotein referred to as chylomicron (CM) is released into the lymph and supplied to the whole body. The TG resynthesis in the small intestinal cells is through two pathways, 2-MG and α-glycerophosphoric acid pathways. Typically, 80% of TG is resynthesized in the 2-MG pathway and the remaining 20% in the 2-plycerophosphoric acid pathways. The TG generated in the 2-MG pathway is utilized for generation of CM in accelerated turnover, and the synthesized CM is secreted into the intestinal lymph and then into blood and is transferred into peripheral tissue (Journal of Clinical Therapeutics and Medicine, vol. 21, No. 2, p. 216, 2005).

Enzymes such as MGATs (acyl-CoA: monoacylglycerol acyltransferases) and DGATs (acyl-CoA: diacylglycerol acyltransferases) are involved specifically in synthesis of TG in a 2-MG pathway. MGATs catalyze a reaction of generation of diacylglycerol by binding between 2-MG generated by lipase and fatty acyl-CoA, whereas DGATs catalyze a reaction of generation of TG by binding between the diacylglycerol generated by the catalytic reaction of the MGATs and fatty acyl-CoA.

Although such MGATs have been suggested to be present in the liver or white adipose tissue (J. Biol. Chem., vol. 259, p. 8934,1984), the cloning of MGAT1 gene, a member of the family of MGATs, has been achieved in recent years, where the gene was isolated, as molecules expressed highly in the kidney, stomach, and white fat and brown fat cells, from a mouse (Proc. Natl. Acad. Sci. USA., vol. 99, p. 8512, 2002). However, although the activity of MGATs was observed significantly in the small intestine, no MGAT1 was expressed in the small intestine, and different molecules belonging to the family of MGATs were thus believed to be present.

Afterward, MGAT2 was cloned through homology search based on the cDNA sequence of MGAT1 by Cao et al., to isolate full-length cDNA from a cDNA library from the mouse small intestine (J. Biol. Chem., vol. 278, p. 13611, 2003). In addition, MGAT3 has been reported to be present in human (J. Biol. Chem., vol. 278, p. 13611, 2003), whereas no MGAT3 has been reported in rodents. The mouse MGAT2 is a 38.6-kDa protein including 334 amino acids, has an N-terminal 40-amino acid signal peptide, includes at least one transmembrane domain, and is expressed strongly in a small intestinal epithelial cell (J. Biol. Chem., vol. 278, p. 13860, 2003). In addition, both human and mouse MGATs2 were reported to include 334 amino acids and have 81% homology in human and mouse amino acid sequences, through the cloning of the human and mouse MGATs2, by Yen et al. (J. Biol. Chem., vol. 278, p. 18532, 2003). The expression pattern of MGAT2 in the small intestine has been exhibited to be similar to that of the site of absorbed lipid (J. Biol. Chem., vol. 279, p. 18878, 2004). In addition, the expression or activity of MGAT2 in the small intestine has been indicated to increase in high-fat diet-induced obesity mice (J. Biol. Chem., vol. 279, p. 18878, 2004) and OLETF rats exhibiting obesity or hypertriglyceridemia (Diabetes Res. Clin. Pract, vol. 57, p. 75, 2002), suggesting that MGAT2 is important for absorbing lipid and is involved in obesity or hypertriglyceridemia.

From the results, an MGAT2 inhibitor is expected to be useful as an agent for treating or preventing obesity, or type 2 diabetes, lipidosis, hypertension, fatty liver, arterial sclerosis, cerebrovascular disorder, coronary artery disease, etc., associated with obesity, through suppressing absorption of lipid.

As a compound having an MGAT2 inhibitory action, for example, a compound represented by a following structure:

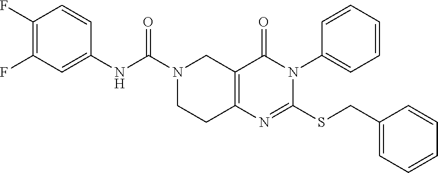

has been disclosed (e.g., see WO 2008/038768). A compound according to the present invention is different from the compound disclosed in WO 2008/038768, in that the compound disclosed in WO 2008/038768 has substituted phenylaminocarbonyl at the 6-position of the 3,4,5,6,7,8-hexahydro-4-oxopyrido[4,3-d]pyrimidine ring whereas the compound according to the present invention has substituted benzimidazolyl. Furthermore, in WO 2008/038768, the substituted phenylaminocarbonyl is not disclosed or suggested to be replaced with substituted benzimidazolyl.

### DISCLOSURE OF THE INVENTION

The present invention provides pyrimidin-4(3H)-one derivatives having MGAT2 inhibitory activity.

The present inventors conducted extensive research for developing a compound having MGAT2 inhibitory activity and found that a compound according to the present invention is efficacious as the compound having the MGAT2 inhibitory activity, and thus accomplished the present invention based on such findings.

Specifically, the present invention relates to a compound represented by a formula (I):

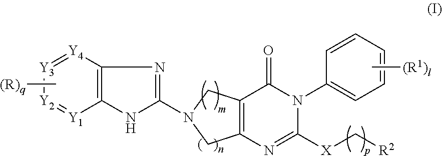

or a pharmaceutically acceptable salt thereof, wherein:
R¹ is selected from the group consisting of:

a lower alkyl group unsubstituted or substituted with 1 to 3 same or different halogen atoms,

a lower alkoxy group unsubstituted or substituted with 1 to 3 same or different halogen atoms, and

a halogen atom;

R² is selected from the group consisting of:

a phenyl group,

a 5- or 6-membered heteroaryl group containing 1 to 3 hetero atom(s) selected from the group consisting of nitrogen atom, sulfur atom and oxygen atom,

a C₃₋₇ cycloalkyl group, wherein one carbon atom of the C₃₋₇ cycloalkyl group may be replaced with nitrogen atom, and

a cyano or lower alkoxycarbonyl group;

wherein said phenyl group, 5- or 6-membered heteroaryl group containing 1 to 3 hetero atom(s) selected from the group consisting of nitrogen atom, sulfur atom and oxygen atom, and C₃₋₇ cycloalkyl group, wherein one carbon atom of the C₃₋₇ cycloalkyl group may be replaced with nitrogen atom, may be substituted with 1 to 3 same or different groups selected from the groups consisting of:

a lower alkyl group unsubstituted or substituted with 1 to 3 same or different halogen atom(s) or hydroxy group,

a lower alkoxy group unsubstituted or substituted with 1 to 3 same or different halogen atom(s),

a lower alkoxy carbonyl group,

a cyano group,

a carbamoyl group,

a mono- or di-lower alkyl carbamoyl group,

a hydroxy group, and

a halogen atom;

R is selected from the group consisting of halogen atom, lower alkyl group or lower alkoxy group;
said lower alkyl group and lower alkoxy group may be substituted with 1 to 3 same or different halogen atom(s);
X is selected from the group consisting of NR', oxygen atom and sulfur atom;
R′ is a lower alkyl group;
each of Y₁, Y₂, Y₃ and Y₄ is all CH, or 1 or 2 of them is nitrogen atom, and the rest are CH;
l is an integer from 0 to 3;
m and n is an integer from 1 or 2;
p is an integer from 0 to 2; and
q is an integer from 1 to 3.

The present invention also relates to an agent for treating and/or preventing hyperlipidemia, diabetes or obesity, containing the compound represented by the formula (I) or the pharmaceutically acceptable salt thereof, as an active ingredient.

The present invention also relates to an MGAT2 inhibitor containing the compound represented by the formula (I) or the pharmaceutically acceptable salt thereof, as an active ingredient.

Furthermore, the present invention relates to a pharmaceutical composition containing the compound represented by the formula (I) and the pharmaceutically acceptable carrier.

The compound (I) according to the present invention or the pharmaceutically acceptable salt thereof has strong MGAT2 inhibitory activity and is thus useful for treating and/or preventing hyperlipidemia, diabetes and obesity.

The meanings of terms as used herein are described below, and the compound according to the present invention is described in further detail.

The term “halogen atom” includes, for example, fluorine, chlorine, bromine and iodine atoms.

The term “lower alkyl” refers to linear or branched C₁₋₆ alkyl, examples of which include methyl, ethyl, propyl, isopropyl, butyl, isobutyl, sec-butyl, tert-butyl, pentyl, isoamyl, neopentyl, isopentyl, 1,1-dimethylpropyl, 1-methylbutyl, 2-methylbutyl, 1,2-dimethylpropyl, hexyl, isohexyl, 1-methylpentyl, 2-methylpentyl, 3-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1-ethylbutyl, 2-ethylbutyl, 1,2,2-trimethylpropyl and 1-ethyl-2-methylpropyl.

The term “lower alkoxy” refers to a group in which the hydrogen atom of hydroxy is substituted with the above-mentioned lower alkyl, examples of which include methoxy, ethoxy, propoxy, isopropoxy, butoxy, sec-butoxy, tert-butoxy, pentyloxy, isopentyloxy, hexyloxy and isohexyloxy.

The term “C₃₋₇ cycloalkyl” includes, for example, cyclopropyl, cyclobutyl, cyclopentyl, cyclohexyl and cycloheptyl.

Specifically compounds according to the present invention are represented by the formula (I):

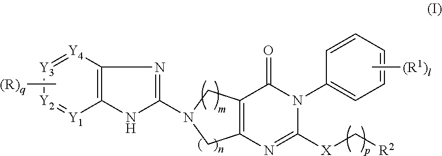

wherein each symbol has the same definition specified above and each symbol used in the formula (I) is described referring to specific examples.

R¹ is each independently selected from the group consisting of:

a lower alkyl group unsubstituted or substituted with 1 to 3 same or different halogen atoms,

a lower alkoxy group unsubstituted or substituted with 1 to 3 same or different halogen atoms, and

a halogen atom.

“Lower alkyl” represented by R¹ is the same as the lower alkyl defined above, of which examples specifically include methyl, ethyl, propoxy and isopropoxy. The lower alkyl is optionally substituted with 1 to 3 same or different halogen atoms.

A halogen atom of the substituent encompasses the same atoms as the halogen atoms defined above, of which examples specifically include fluorine, chlorine, bromine and iodine atoms.

“Lower alkyl optionally substituted with 1 to 3 same or different halogen atoms” represented by R¹ specifically encompasses fluoromethyl, chloromethyl, bromomethyl, difluoromethyl and trifluoromethyl.

“Lower alkoxy” represented by R¹ is the same as the lower alkoxy defined above, examples of which include methoxy, ethoxy, propoxy and isopropoxy.

The lower alkoxy is optionally substituted with 1 to 3 same or different halogen atoms.

A halogen atom of the substituent encompasses the same atoms as the halogen atoms defined above, of which examples specifically include fluorine, chlorine, bromine and iodine atoms.

“Lower alkoxy optionally substituted with 1 to 3 same or different halogen atoms” represented by R¹ specifically encompasses fluoromethoxy, chloromethoxy, bromomethoxy, difluoromethoxy and trifluoromethoxy.

“Halogen atom” represented by R¹ encompasses the same atoms as the halogen atoms defined above, of which examples specifically include fluorine, chlorine, bromine and iodine atoms.

R² is selected from the group consisting of:

a phenyl group,

a 5- or 6-membered heteroaryl group containing 1 to 3 hetero atom(s) selected from the group consisting of nitrogen atom, sulfur atom and oxygen atom,

a C₃₋₇ cycloalkyl group, wherein one carbon atom of the C₃₋₇ cycloalkyl group may be replaced with nitrogen atom, and

a cyano or lower alkoxycarbonyl group;

“5- or 6-membered heteroaryl having 1-3 hetero atoms selected from the group consisting of nitrogen, sulfur and oxygen atoms, contained within a ring” represented by R² specifically encompasses, e.g., oxadiazolyl, thiadiazolyl, pyridinyl, pyrazinyl, pyridazinyl, pyrimidinyl, pyrrolyl, imidazolyl and pyrazolyl.

“C₃₋₇ cycloalkyl, which is composed of carbon atoms of which one is optionally substituted with a nitrogen atom” represented by R² specifically encompasses, e.g., cyclopropyl, cyclobutyl, cyclopentyl, cyclohexyl, cycloheptyl, piperidinyl and pyrrolidinyl.

“Phenyl, 5- or 6-membered heteroaryl having 1-3 hetero atoms selected from the group consisting of nitrogen, sulfur and oxygen atoms, contained within a ring, and C₃₋₇ cycloalkyl, which is composed of carbon atoms of which one is optionally substituted with a nitrogen atom” represented by R² is optionally substituted with 1 to 3 same or different groups selected from the group consisting of lower alkyl optionally substituted with 1 to 3 same or different halogen atoms or hydroxy groups, lower alkoxy which may be substituted with 1 to 3 same or different halogen atoms, lower alkoxycarbonyl, cyano, carbamoyl, mono- or di-lower alkylcarbamoyl, hydroxyl and halogen atoms.

“Lower alkyl” of the substituent refers to the same groups as the lower alkyl defined above, of which examples specifically include methyl, ethyl, propyl and isopropyl.

The lower alkyl is optionally substituted with 1 to 3 same or different halogen atoms or hydroxy groups.

“Lower alkyl substituted with 1 to 3 same or different halogen atoms” of the substituent specifically encompasses, e.g., fluoromethyl, chloromethyl, bromomethyl, difluoromethyl and trifluoromethyl.

“Lower alkyl substituted with hydroxy” of the substituent specifically encompasses, e.g., hydroxymethyl, hydroxyethyl and hydroxypropyl.

“Lower alkoxy” of the substituent refers to the same groups as the lower alkoxy defined above, of which examples specifically include methoxy, ethoxy, propoxy and isopropoxy.

The lower alkoxy is optionally substituted with 1 to 3 same or different halogen atoms.

A halogen atom of the substituent encompasses the same groups as the halogen atoms defined above, of which examples specifically include fluorine, chlorine, bromine and iodine atoms.

“Lower alkoxy substituted with 1 to 3 same or different halogen atoms” of the substituent specifically encompasses, e.g., fluoromethoxy, chloromethoxy, bromomethoxy, difluoromethoxy and trifluoromethoxy.

“Lower alkoxycarbonyl” of the substituent refers to a group having the above-defined lower alkoxy bound to carbonyl and specifically encompasses, e.g., methoxycarbonyl, ethoxycarbonyl, propoxycarbonyl and isopropoxycarbonyl.

“Mono-lower alkylcarbamoyl” of the substituent refers to carbamoyl mono-substituted with the above-defined lower alkyl and specifically encompasses, e.g., methylcarbamoyl, ethylcarbamoyl, propylcarbamoyl and isopropylcarbamoyl.

“Di-lower alkylcarbamoyl” of the substituent refers to carbamoyl di-substituted with the above-defined same or different lower alkyl and specifically encompasses, e.g., dimethylcarbamoyl, diethylcarbamoyl, dipropylcarbamoyl, diisopropylcarbamoyl and ethylmethylcarbamoyl.

“Halogen atom” of the substituent refers to the same atom the halogen atoms defined above, of which examples specifically include fluorine, chlorine, bromine and iodine atoms.

“Lower alkoxycarbonyl” represented by R² refers to a group having the above-defined lower alkoxy bound to carbonyl and specifically encompasses, e.g., methoxycarbonyl, ethoxycarbonyl, propoxycarbonyl and isopropoxycarbonyl.

R is each independently halogen atom, lower alkyl or lower alkoxy, said lower alkyl and lower alkoxy is optionally substituted with 1 to 3 same or different halogen atoms.

“Halogen atom” represented by R refers to the same atom as the halogen atoms defined above, of which examples specifically include fluorine, chlorine, bromine and iodine atoms.

“Lower alkyl” represented by R refers to the same groups as the lower alkyl defined above, of which examples specifically include methyl, ethyl, propyl and isopropyl.

The lower alkyl may be substituted with 1 to 3 same or different halogen atoms.

“Lower alkyl substituted with 1 to 3 same or different halogen atoms” represented by R refers to the above-defined lower alkyl substituted with 1 to 3 same or different halogen atoms and specifically encompasses, e.g., fluoromethyl, chloromethyl, bromomethyl, difluoromethyl and trifluoromethyl.

“Lower alkoxy” represented by R refers to the same groups as the lower alkoxy defined above, of which examples specifically include methoxy, ethoxy, propoxy and isopropoxy.

The lower alkoxy is optionally substituted with 1 to 3 same or different halogen atoms.

“Lower alkoxy substituted with 1 to 3 same or different halogen atoms” represented by R refers to the above-defined lower alkoxy substituted with 1 to 3 same or different halogen atoms and specifically encompasses, e.g., fluoromethoxy, chloromethoxy, bromomethoxy, difluoromethoxy and trifluoromethoxy.

R is preferably trifluoromethyl or a fluorine atom.

X represents a group selected from the group consisting of NR′ and oxygen and sulfur atoms, wherein R′ refers to lower alkyl.

“Lower alkyl” represented by R′ encompasses the same groups as the lower alkyl defined above, of which examples specifically include methyl, ethyl, propyl and isopropyl.

X is preferably an oxygen or sulfur atom.

All Y₁, Y₂, Y₃ and Y₄ represent CH, or one or two of Y₁ to Y₄ are nitrogen atoms and the others represent CH.

All Y₁, Y₂, Y₃ and Y₄ are preferably CH.

An integer of from 0 to 3 is represented by 1, wherein 1 is preferably 1.

An integer of 1 or 2 is represented by m and n each.

The case in which m is 1 and n is 2 or the case in which m is 2 and n is 1 is preferred.

An integer of from 0 to 2 is represented by p, wherein p is 0 or 1.

An integer of from 1 to 3 is represented by q.

In accordance with a preferred embodiment, any aspects of R, R¹, R², X, Y₁, Y₂, Y₃, Y₄, p, q, l, m and n as described above may be combined.

Compounds according to the present invention include, e.g., compounds as described in Examples or pharmaceutically acceptable salts thereof, but are not limited thereto.

A process of producing the compound according to the present invention will now be described.

The compound (I) according to the present invention can be produced, e.g., by the following process:

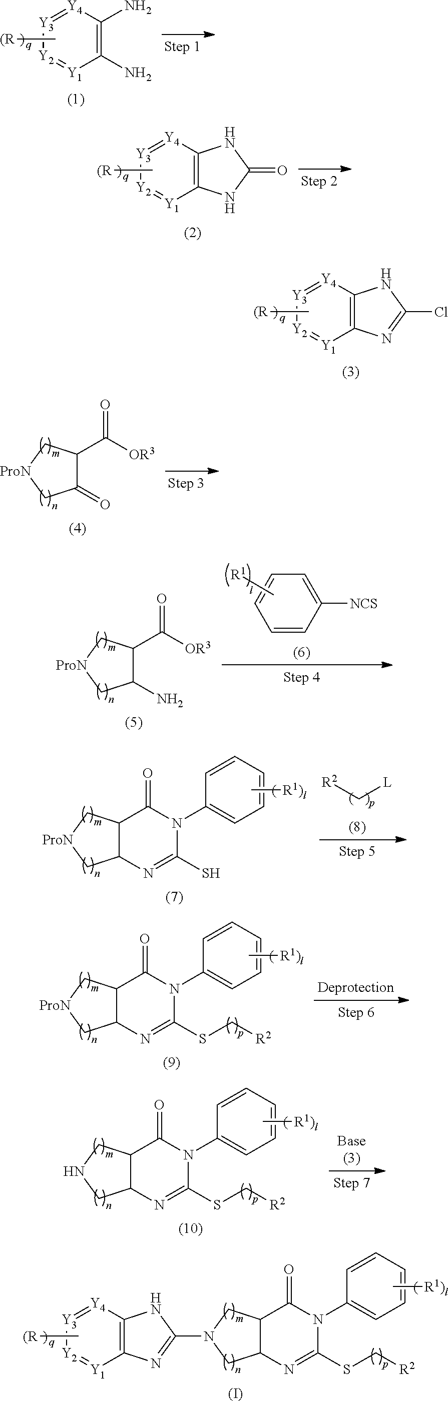

wherein Pro represents a protective group for an amino group; R³ represents a lower alkyl group; L represents a leaving group; and the other symbols have the same definitions specified above, processes described in Examples or other processes known in the art.

### Step 1

This step is a process of producing a compound (2) by reacting a compound (1) with CDI (carbonyldiimidazole).

An amount of CDI used in this step is typically 1-10 equivalents, preferably 1-2 equivalents, relative to 1 equivalent of the compound (1).

The reaction temperature is typically 0-100° C., preferably from 0° C. to room temperature.

The reaction time is typically 5 minutes to 24 hours, preferably 0.5-2 hours.

Unless interfering with the reaction, any solvent may be used, examples of which include THF (tetrahydrofuran), DMF (dimethylformamide) and chloroform.

The compound (2) thus may be isolated and purified with well-known separation and purification methods such as concentration, concentration under reduced pressure, reprecipitation, solvent extraction, crystallization and chromatography, or subjected to the next step without isolation or purification.

### Step 2

This step is a process for producing a compound (3) by reacting the compound (2) with phosphorous oxychloride.

An amount of phosphorous oxychloride used in this step is typically 1-100 equivalents, preferably 1-3 equivalents, relative to 1 equivalent of the compound (2).

The reaction temperature is typically 0-150° C., preferably 0-80° C.

The reaction time is typically 0.5-24 hours, preferably 0.5-2 hours.

Unless interfering with the reaction, any solvent may be used, examples of which include benzene and toluene. The reaction may be also carried out without using any solvent.

The compound (3) thus obtained may be isolated and purified with well-known separation and purification methods such as concentration, concentration under reduced pressure, reprecipitation, solvent extraction, crystallization and chromatography, or subjected to the next step without isolation or purification.

### Step 3

This step is a process for producing a compound (5) by reacting the compound (4) with ammonia.

R³ represents lower alkyl and encompasses, e.g., methyl and ethyl.

An amount of ammonia used in this step is typically 1-100 equivalents, preferably 1-10 equivalents, relative to 1 equivalent of the compound (4).

The reaction temperature is typically 0-100° C., preferably 20-80° C.

The reaction time is typically 0.5-24 hours, preferably 0.5-2 hours.

Unless interfering with the reaction, any solvent may be used, examples of which include methanol and ethanol.

The compound (5) thus obtained may be isolated and purified with well-known separation and purification methods such as concentration, concentration under reduced pressure, reprecipitation, solvent extraction, crystallization and chromatography, or subjected to the next step without isolation or purification.

### Step 4

This step is a process for producing a compound (7) by reacting the compound (5) with a compound (6).

An amount of the compound (6) as used is typically 1-10 equivalents, preferably 1-2 equivalents, relative to 1 equivalent of the compound (5).

The reaction temperature is typically 0-120° C., preferably from room temperature to 80° C.

The reaction time is typically 0.5-24 hours, preferably 0.5-2 hours.

Unless interfering with the reaction, any solvent may be used, examples of which include DMF (dimethylformamide), pyridine, toluene and THF.

The compound (7) thus obtained may be isolated and purified with well-known separation and purification methods such as concentration, concentration under reduced pressure, reprecipitation, solvent extraction, crystallization and chromatography, or subjected to the next step without isolation or purification.

### Step 5

This step is a process for producing a compound (9) by reacting the compound (7) with a compound (8).

An amount of the compound (8) as used is typically 1-10 equivalents, preferably 1-2 equivalents, relative to 1 equivalent of the compound (7).

Leaving groups represented by L in the compound (8) include, e.g., halogen atoms such as chlorine and bromine.

The reaction temperature is typically 0-150° C., preferably 0-80° C.

The reaction time is typically 0.5-24 hours, preferably 0.5-2 hours.

Unless interfering with the reaction, any solvent may be used, examples of which include DMF, THF and dioxane.

The compound (9) thus obtained may be isolated and purified with well-known separation and purification methods such as concentration, concentration under reduced pressure, reprecipitation, solvent extraction, crystallization and chromatography, or subjected to the next step without isolation or purification.

### Step 6

This step is a process for producing a compound (10) by removing a protective group Pro for the amino group of the compound (9).

The protective group for amino can be removed by a method as described in documents (e.g., T. W. Green: Protective Groups in Organic Synthesis, Second Edition, John Wiley & Sons (1991)), methods equivalent thereto or combinations of these with other methods known in the art. Specifically, for example, when a Boc group is used as a protective group for amino, the protective group can be removed by TFA (trifluoroacetic acid).

The compound (10) thus obtained may be isolated and purified with well-known separation and purification methods such as concentration, concentration under reduced pressure, reprecipitation, solvent extraction, crystallization and chromatography, or subjected to the next step without isolation or purification.

### Step 7

This step is a process for producing a compound (I) according to the present invention by reacting the compound (3) with the compound (10) in the presence of base.

Bases as used include, e.g., DIEA (diisopropylethylamine), TEA (triethylamine) and DBU.

This reaction may be also carried out by microwaving a reaction system.

The reaction temperature is typically 0-200° C., preferably from room temperature to 180° C.

The reaction time is typically 10 minutes to 8 hours, preferably 10 minutes to 0.5 hour.

Unless interfering with the reaction, any solvent may be used, examples of which include dioxane, toluene, DMF, THF and acetonitrile.

The compound (I) according to the present invention thus obtained may be isolated and purified in well-known separation and purification method such as concentration, vacuum concentration, reprecipitation, solvent extraction, crystallization and chromatography.

The pyrimidin-4(3H)-one derivative in accordance with the present invention may be present as a pharmaceutically acceptable salt, which may be produced according to usual methods using the compound (I).

The acid-addition salts include, for example, hydrohalides such as hydrochlorides, hydrofluorides, hydrobromides, hydroiodides; inorganic acid salts such as nitrates, perchlorates, sulfates, phosphates, carbonates; lower alkylsulfonates such as methanesulfonates, trifluoromethanesulfonates, ethanesulfonates; arylsulfonates such as benzenesulfonates, p-toluenesulfonates; organic acid salts such as fumarates, succinates, citrates, tartrates, oxalates, maleates; other organic acid-addition salts with amino acid such as glutamates, aspartates.

When the compounds of the invention have an acid group in the molecule, for example, when they have a carboxyl group, then the compounds may be processed with a base so as to convert them into the corresponding pharmaceutically-acceptable salts.

The base-addition salts include, for example, alkali metal salts with sodium or potassium; alkaline earth metal salts with calcium or magnesium; ammonium salts; organic base-addition salts with guanidine, triethylamine, dicyclohexylamine, etc.

In addition, the compounds of the invention may also be in any other form of hydrates or solvates of their free compounds or their salts.

Conversely, conversion from a salt or an ester into a free compound may also be accomplished according to a standard method.

Depending on the type of the substituents therein, the compounds of the invention include stereoisomers and tautomers such as optical isomers, diastereomeric isomers and geometrical isomers. Needless-to-say, the compounds of the invention include all these isomers. Further needless-to-say, the compounds of the invention include all mixtures of such isomers.

In producing medicines for prevention and remedy for type II diabetes or diseases or symptoms associated with it, the compounds of the formula (I) of the invention may be combined with carrier substances.

The dose of the compounds of the formula (I) of the invention for prevention or remedy for diseases naturally varies, depending on the property of the symptom to which the treatment is directed, the specific compound selected for it and the administration route.

In addition, the dose also varies depending on the age, the body weight and the sensitivity of patients. In general, the daily dose for one-time or plural-times administration may be from about 0.001 mg/kg-body weight to about 100 mg/kg-body weight, preferably from about 0.01 mg/kg-body weight to about 50 mg/kg-body weight, even more preferably from about 0.1 mg/kg-body weight to about 10 mg/kg-body weight. As the case may be, administration of a dose over the range may be necessary.

An example of a suitable dose for oral administration is described. The daily dose for one-time or two- to four-times administration may be at least from about 0.01 mg to at most 2.0 g. Preferably, the daily administration frequency is once or twice a day, and the daily dose is from about 1.0 mg to about 200 mg. More preferably, the daily dose is from about 10 mg to 100 mg for one-time administration a day.

For intravenous administration or oral administration, a typical dose of the compound (1) may be from about 0.001 mg/day/kg-body weight to about 100 mg/day/kg-body weight (preferably from 0.01 mg/day/kg-body weight to about 10 mg/day/kg-body weight), more preferably from about 0.1 mg/day/kg-body weight to 10 mg/day/kg-body weight.

As so mentioned hereinabove, the pharmaceutical composition of the invention comprises the compound of the formula (I) and a pharmaceutically-acceptable carrier. The term “composition” is meant to contain not only a product produced by directly or indirectly combining, hybridizing or aggregating 2 or more ingredients, a product produced as a result of dissociation of one or more ingredients, or a compound produced as a result of reaction or interaction of different types of ingredients, but also an active and inactive ingredient of constituting a carrier (pharmaceutically-acceptable vehicle).

As combined with a pharmaceutically-acceptable carrier, the composition of the invention preferably contains the compound of the formula (I) in an amount effective for remedy and prevention of type II diabetes and for retardation of the onset of the disease.

For administering the effective dose of the compound of the invention to mammals, especially to humans, employable is any suitable administration route. For example, the route may be oral administration, rectal administration, local administration, intravenous administration, ophthalmic administration, lung administration or nasal administration. Examples of the administration forms are tablets, troches, powders, suspensions, solutions, capsules, creams, aerosols. Preferred are oral tablets.

In preparing oral compositions, any ordinary pharmaceutical media can be used. Their examples are water, glycol, oil, alcohol, fragrant additives, preservatives, colorants. In preparing liquid compositions for oral administration, for example, mentioned are suspensions, elixirs and solutions. Their carriers are, for example, starch, sugar, microcrystalline cellulose, diluent, granulating promoter, lubricant, binder, disintegrator. In preparing solid compositions for oral administration, for example, mentioned are powders, capsules and tablets. Above all, such solid compositions for oral administration are preferred.

In view of the easiness in their administration, tablets and capsules are the most advantageous forms for oral administration. If desired, the tablets may be coated according to standard aqueous or non-aqueous coating techniques.

In addition to the above-mentioned ordinary administration modes for them, the compounds of the formula (I) may also be administered according to controlled release systems and/or controlled delivery systems, for example, as in U.S. Pat. Nos. 3,845,770, 3,916,899, 3,536,809, 3,598,123, 3,630,200 and 4,008,719.

The pharmaceutical composition of the invention suitable for oral administration includes capsules, cashews and tablets that contain a predetermined amount of the active ingredient in the form of powders or granules thereof, or in the form of water-soluble liquids, water-insoluble liquids, oil-in-water emulsions or water-in-oil emulsions thereof. These compositions may be prepared in any pharmaceutical methods, and all the methods include a process of combining the active ingredient with a carrier of one or more necessary ingredients.

In general, the active ingredient is uniformly and fully mixed with a liquid carrier, or a well-separated solid carrier or with both the two, and then, if desired, the product is shaped into suitable forms to prepare the composition. For example, tablets are produced through compression and shaping, optionally along with one or more side components. Using a suitable machine, compressed tablets may be produced by mixing the active ingredient optionally with binder, lubricant, inert vehicle, surfactant or dispersant and compressing the resulting mix in any desired manner into powders or granules.

Shaped tablets may be prepared by shaping a mixture of a powdery wet compound and an inert liquid diluent, using a suitable machine.

Preferably, the tablets each contain from about 1 mg to 1 g of the active ingredient; and the cashews and the capsules each contain from about 1 mg to 500 mg of the active ingredient.

Examples of the administration modes of the compounds of the formula (I) for pharmaceutical use are as follows:

TABLE 1
Suspension for Injection (I. M.)

| | mg/ml |
|---|---|
| compound of formula (I) | 10 |
| methyl cellulose | 5.0 |
| Tween 80 | 0.5 |
| benzyl alcohol | 9.0 |
| Benzalkonium chloride | 1.0 |

water for injection added to make 1.0 ml

TABLE 2

| Tablets | mg/tablet |
|---|---|
| compound of formula (I) | 25 |
| methyl cellulose | 415 |
| Tween 80 | 14.0 |
| benzyl alcohol | 43.5 |
| magnesium stearate | 2.5 |
| total | 500 mg |

TABLE 3

| Capsules | mg/capsule |
|---|---|
| compound of formula (I) | 25 |
| lactose powder | 573.5 |
| magnesium stearate | 1.5 |
| total | 600 mg |

TABLE 4

| Aerosol | per one container | |
|---|---|---|
| compound of formula (I) | 24 | mg |
| lecithin, NF Liq. Conc. | 1.2 | mg |
| trichlorofluoromethane, NF | 4.025 | g |
| dichlorodifluoromethane, NF | 12.15 | g |

The compounds of the formula (I) may be used, as combined with any other drugs usable not only for type II diabetes-associated diseases or symptoms but also for remedy/prevention/retardation of the onset of type II diabetes. The additional drugs may be administered in any administration route and dose generally employed in the art, simultaneously with or separately from the compound of the formula (I).

In case where the compound of the formula (I) is used along with one or more other drugs, then a pharmaceutical composition comprising the compound of the formula (I) and the additional drug is preferred. Accordingly, the pharmaceutical composition of the invention may comprise not only the compound of the formula (I) but also one or more such active ingredients. Examples of the active ingredients that may be combined with the compounds of the formula (I) are mentioned below. The list below is not all inclusive. These may be separately administered or may be administered simultaneously as contained in the same pharmaceutical composition.

(a) other MGAT2 inhibitor
(b) glucokinase activators,
(c) bis-guanides (e.g., buformin, metoformin, fenformin,),
(d) PPAR agonists (e.g., triglytazon, pioglytazon, nosiglytazon),
(e) insulin,
(f) somatostatin,
(g) α-glucosidase inhibitors (e.g., boglybose, miglytol, acarbose),
(h) insulin secretion promoters (e.g., acetohexamide, calbutamide, chlorpropamide, glybomlide, glycrazide, glymerpiride, glypidide, glyquidine, glysoxepide, glyburide, glyhexamide, glypinamide, fenbutamide, trazamide, tolbutamide, tolcyclamide, nateglynide, repaglynide),
(i) DPP-IV inhibitors (dipeptidyl peptidase IV inhibitors, e.g., Sitagliptin). The weight ratio of the compound of the formula (I) to the second active ingredient may vary within a broad range, and depends on the effective amount of the individual active ingredients. Accordingly, for example, when the compound of the formula (I) is combined with a PPAR agonist, then the weight ratio of the compound of the formula (I) to the PPAR agonist may be generally from about 1000/1 to 1/1000, preferably from about 200/1 to 1/200. The combination of the compound of the formula (I) and the other active ingredient may be within the above-mentioned range. And in any case, the effective dose of the individual ingredients should be used.

The compound according to the present invention or a pharmaceutically acceptable salt thereof has strong MGAT2 inhibitory activity and is thus useful for treating and/or preventing hyperlipidemia, diabetes and obesity.

It should be understood by those skilled in the art that various modifications, combinations, sub-combinations and alterations may occur depending on design requirements and other factors insofar as they are within the scope of the appended claims or the equivalents thereof.

### EXAMPLES

The present invention is described below in more detail referring to Formulation Examples, Examples and Reference Examples, but is not limited thereto.

### Formulation Example 1

Ten parts of the compound in accordance with Example 1, 15 parts of heavy magnesium oxide and 75 parts of lactose are blended uniformly to prepare a powder having a particle size of 350 μm or less in powder or granular form. The powder is charged in a capsule container to form a capsule.

### Formulation Example 2

After uniformly blending 45 parts of the compound in accordance with Example 1, 15 parts of starch, 16 parts of lactose, 21 parts of crystalline cellulose, 3 parts of poly vinyl alcohol and 30 parts of distilled water, the blend is crushed into granules, which are dried and then sieved to form granules having a particle diameter of 177-1410 μm.

### Formulation Example 3

After preparing granules in the same manner as in Formulation Example 2, 3 parts of calcium stearate is added to 96 parts of the granules, and the mixture is compression-molded to prepare tablets having a diameter of 10 mm.

### Formulation Example 4

To 90 parts of the granules prepared by the method described in Formulation Example 2 is added 10 parts of crystalline cellulose and 3 parts of calcium stearate, and the mixture is compression-molded to form tablets having a diameter of 8 mm, to which a syrup gelatin/precipitated calcium carbonate suspension is added to prepare sugar-coated tablets.

Wakogel (registered trademark) C-300, made by Wako Pure Chemical Industries Ltd., or KP-Sil (Registered Trademark) Silica prepacked column, made by Biotage, was used for the silica gel column chromatography in Examples. Kieselgel™ 60 F₂₅₄, Art. 5744, made by Merck & Co., was used for preparative thin layer chromatography. Chromatorex (registered trademark) NH (100-250 mesh or 200-350 mesh), made by Fuji Silysia Chemical Ltd., was used for basic silica gel column chromatography.

¹H-NMR was measured using JEOL AL400 (400 MHz), Mercury (400 MHz) and Inova (400 MHz), made by Varian, using tetramethylsilane as a standard substance. In addition, the mass spectra were measured by electrospray ionization (ESI) or atmospheric pressure chemical ionization (APCI) using Micromass ZQ made by Waters.

The meanings of the abbreviations in Examples are shown below.

i-Bu=isobutyl
n-Bu=n-butyl
t-Bu=tert-butyl
Boc=tert-butoxycarbonyl
Me=methyl
Et=ethyl
Ph=phenyl
i-Pr=isopropyl
n-Pr=n-propyl
CDCl₃=heavy chloroform
CD₃OD=heavy methanol
DMSO-d₆=heavy dimethylsulfoxide

The meanings of the abbreviations in the nuclear magnetic resonance spectra are shown below.

s=singlet
d=doublet
dd=double doublet
dt=double triplet
ddd=double double doublet
Sept=septet
t=triplet
m=multiplet
br=broad
brs=broad singlet
q=quartet
J=coupling constant
Hz=hertz

### Example 1

### Production of 6-(5,6-difluoro-1H-benzimidazol-2-yl)-3-phenyl-2-[(phenylmethyl)thio]-5,6,7,8-tetrahydropyrido[4,3-d]pyrimidin-4(3H)-one

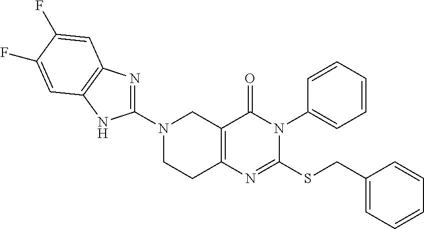

3-phenyl-2-[(phenylmethyl)thio]-5,6,7,8-tetrahydropyrido[4,3-d]pyrimidin-4(3H)-one hydrochloride, 2-chloro-5,6-difluorobenzimidazole and diisopropylethylamine (95 mg, 0.73 mmol) were suspended in 3 ml of acetonitrile, and the suspension was reacted at 180° C. for 2 hours with a microwave device. Then, the reaction solution was concentrated and purified by silica gel column chromatography, to yield the above-mentioned title compound as a brown solid.

¹H-NMR (DMSO-D₆) δ: 11.79 (1H, s),7.51-7.45 (3H, m),7.34-7.27 (4H, m),7.25-7.08 (5H, m),4.29 (2H, brs), 4.23 (2H, brs),3.85-3.81 (2H, brm),2.82-2.76 (2H, brm).

ESI-MS Found: m/z 502 [M+H]+

### Example 2

### Production of 6-(5-trifluoromethyl-1H-benzimidazol-2-yl)-3-phenyl-2-[(phenylmethyl)thio]-5,6,7,8-tetrahydropyrido[4,3-d]pyrimidin-4(3H)-one

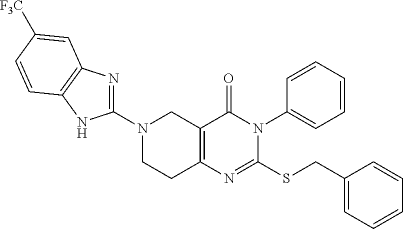

The above-mentioned title compound was obtained as a colorless solid from 2-chloro-6-trifluoromethyl-1H-benzimidazole by the same procedure as in Example 1.

¹H-NMR (DMSO-D₆) δ: 12.04-11.97 (1H, m),7.53-7.48 (3H, m), 7.38-7.18 (10H, m), 4.39 (2H, s), 4.27 (2H, s), 3.93-3.88 (2H, brm), 2.86-2.81 (2H, brm).

ESI-MS Found: m/z 534 [M+H]+

### Example 3

### Production of 3-phenyl-2-[(pyridin-3-ylmethyl)thio]-6-[5-(trifluoromethyl)-1H-benzimidazol-2-yl]-5,6,7,8-tetrahydropyrido[4,3-d]pyrimidin-4(3H)-one

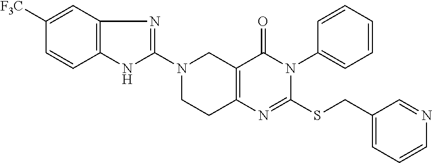

The above-mentioned title compound was obtained as a colorless solid from 2-chloro-6-trifluoromethyl-1H-benzimidazole and a compound in accordance with Reference Example 4 by the same procedure as in Example 1.

¹H-NMR (CDCl₃) δ: 2.92 (2H, t, J=5.9 Hz), 4.01 (2H, t, J=5.9 Hz), 4.21 (2H, s), 4.42 (2H, s), 7.11-7.14 (2H, m), 7.17 (1H, dd, J=7.8, 4.7 Hz), 7.24-7.32 (6H, m), 7.57-7.61 (1H, m), 7.57 (1H, s), 8.44 (1H, dd, J=4.7, 1.6 Hz), 8.56 (1H, d, J=2.0 Hz).

ESI-MS Found: m/z 535.4 [M+H]+

### Example 4

### Production of 3-phenyl-2-[(pyridazin-4-ylmethyl)thio]-6-[5-(trifluoromethyl)-1H-benzimidazol-2-yl]-5,6,7,8-tetrahydropyrido[4,3-d]pyrimidin-4(3H)-one

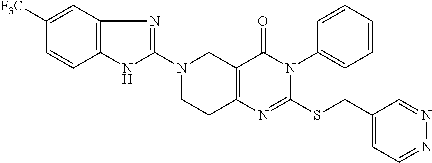

The above-mentioned title compound was obtained as a colorless solid from 2-chloro-6-trifluoromethyl-1H-benzimidazole and a compound in accordance with Reference Example 6 by the same procedure as in Example 1.

¹H-NMR (CDCl₃) δ: 2.88 (2H, t, J=5.7 Hz), 3.99 (2H, t, J=5.7 Hz), 4.15 (2H, s), 4.41 (2H, s), 7.13-7.16 (2H, m), 7.24-7.35 (6H, m), 7.39 (1H, dd, J=5.3, 2.3 Hz), 9.08 (1H, dd, J=5.3, 1.0 Hz), 9.17-9.19 (1H, m).

ESI-MS Found: m/z 536.4 [M+H]+

Compounds described below can be produced by the above-mentioned general production processes, the processes described in Examples 1-4, processes equivalent thereto or combinations of these with usual processes.

TABLE 5

| | | Parent ion |
|---|---|---|
| Example | | m/z [M + H]+ |
| No. | Structure | ESI-MS |
| 5 | 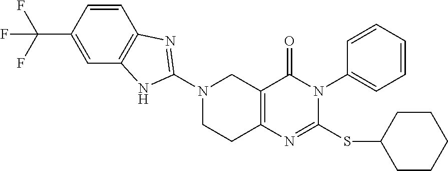 | 526.4 |
| 6 | 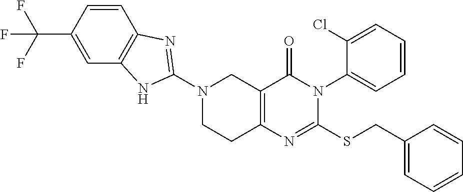 | 568.3 |
| 7 | 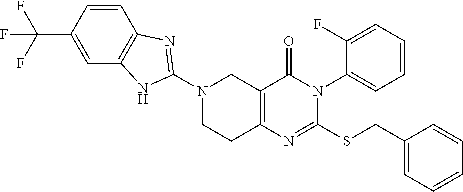 | 552.1 |
| 8 | 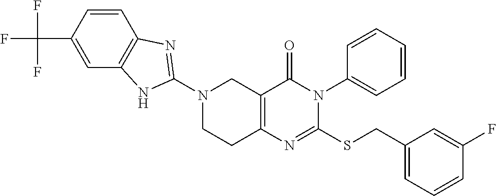 | 522.1 |
| 9 | 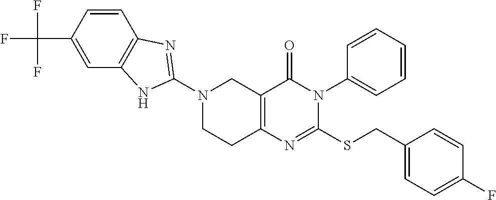 | 522.1 |
| 10 | 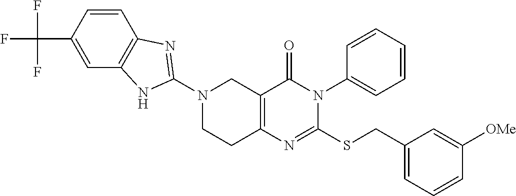 | 564.1 |

TABLE 6

| | | |
|---|---|---|
| 11 | 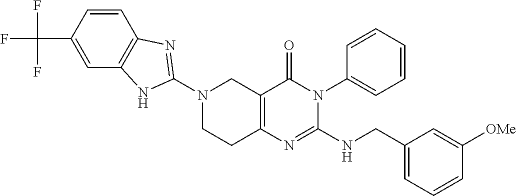 | 547.1 |
| 12 | 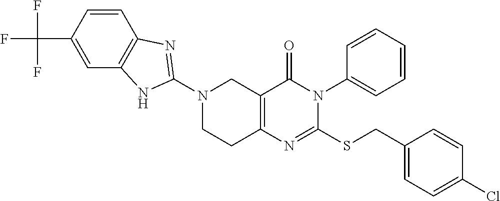 | 568.1 |
| 13 | 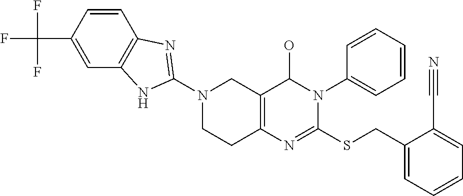 | 559.1 |
| 14 | 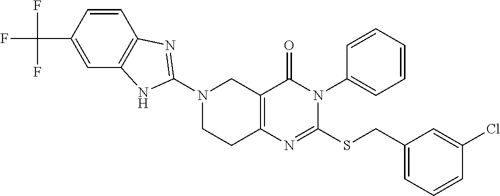 | 568.1 |
| 15 | 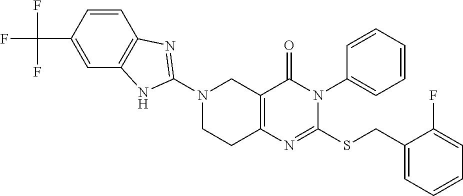 | 552.0 |
| 16 | 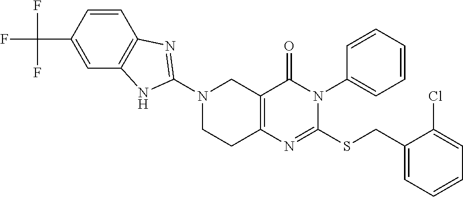 | 568.1 |

TABLE 7

| | | |
|---|---|---|
| 17 | 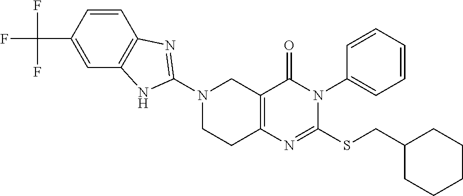 | 540.2 |
| 18 | 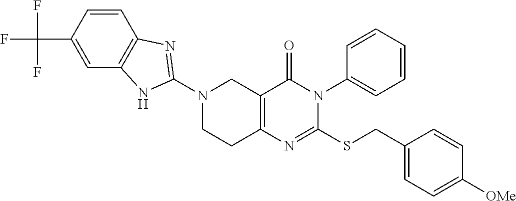 | 564.1 |
| 19 | 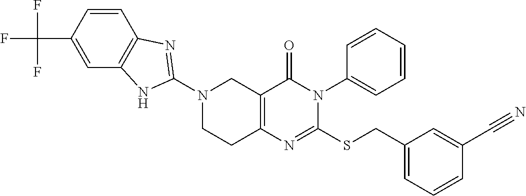 | 559.1 |
| 20 | 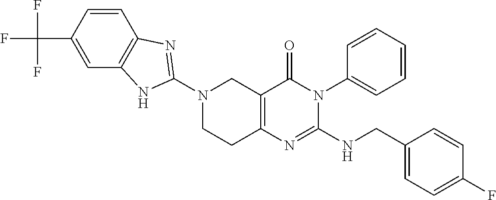 | 535 |
| 21 | 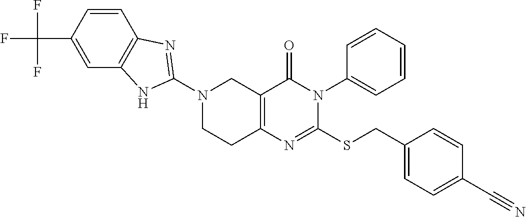 | 559.2 |
| 22 | 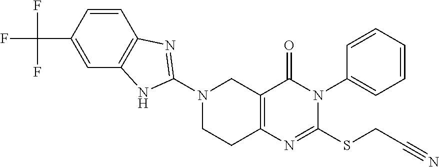 | 483.0 |

TABLE 8

| | | |
|---|---|---|
| 23 | 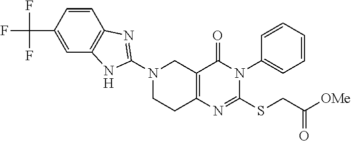 | 516.1 |
| 24 | 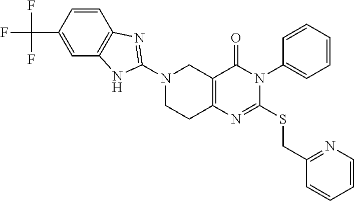 | 535.2 |
| 25 | 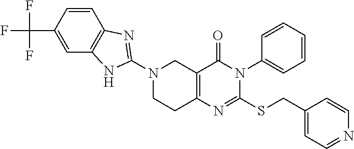 | 535.2 |
| 26 | 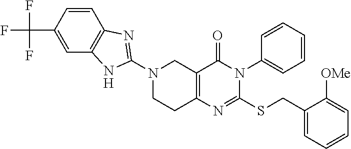 | 564.2 |
| 27 | 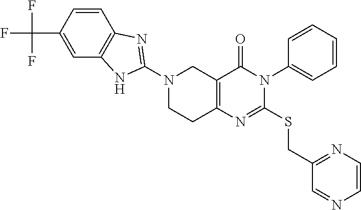 | 536.1 |
| 28 | 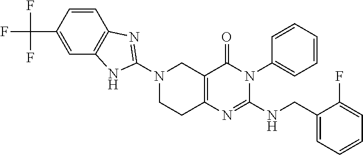 | 535.1 |

TABLE 9

| | | |
|---|---|---|
| 29 | 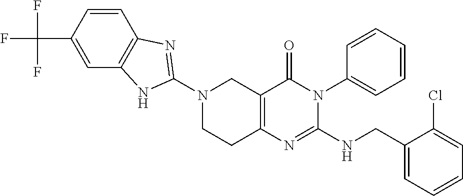 | 551.1 |
| 30 | 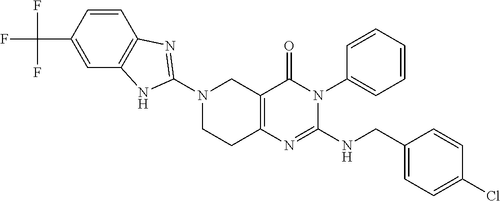 | 551.1 |
| 31 | 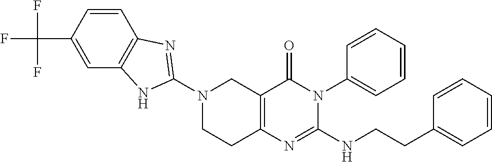 | 531.2 |
| 32 | 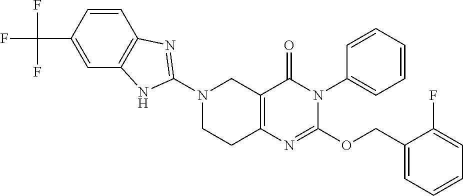 | 536.1 |
| 33 | 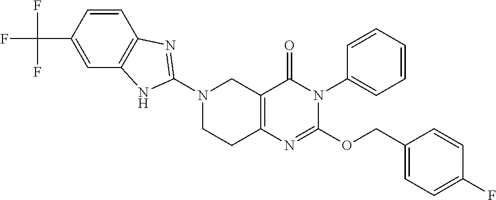 | 536.2 |
| 34 | 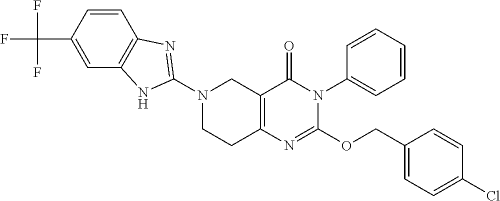 | 552.1 |

TABLE 10

| | | |
|---|---|---|
| 35 | 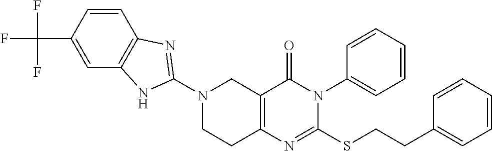 | 548.1 |
| 36 | 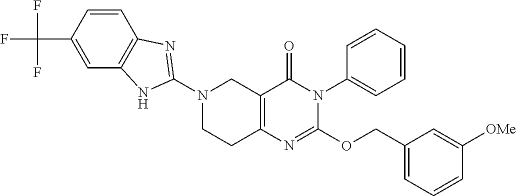 | 548.2 |
| 37 | 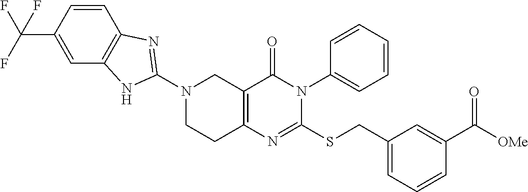 | 592.1 |
| 38 | 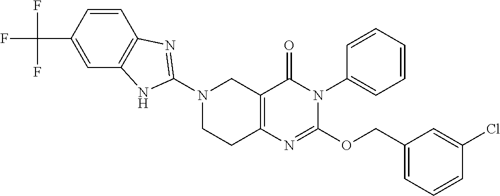 | 552.1 |
| 39 | 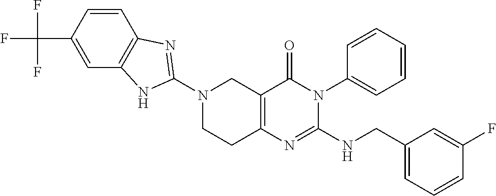 | 535.1 |
| 40 | 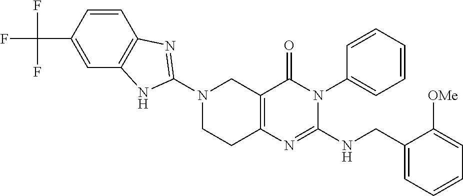 | 547.2 |

TABLE 11

| | | |
|---|---|---|
| 41 | 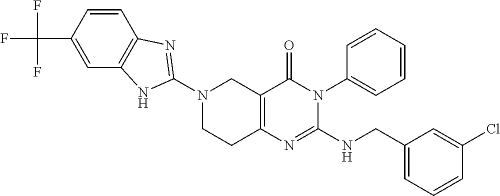 | 551.2 |
| 42 | 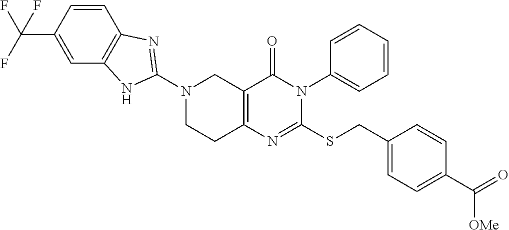 | 592.2 |
| 43 | 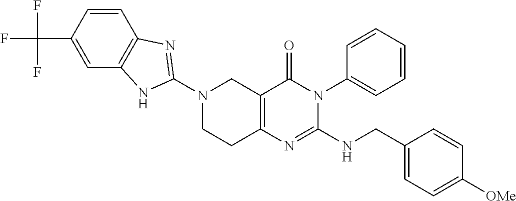 | 547.1 |
| 44 | 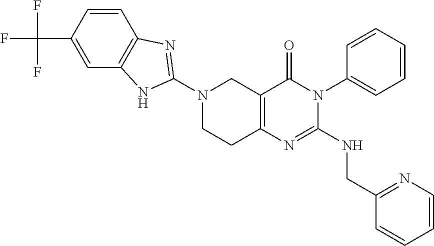 | 518.3 |
| 45 | 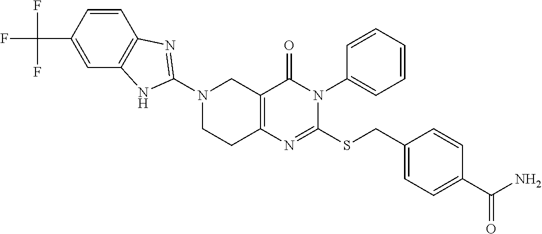 | 577.3 |
| 46 | 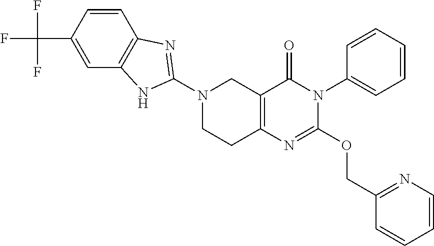 | 519.2 |

TABLE 12

| | | |
|---|---|---|
| 47 | 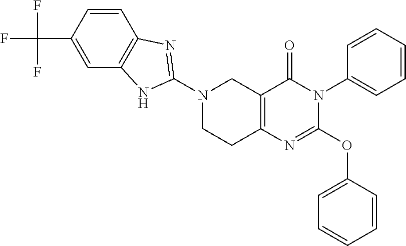 | 504.2 |
| 48 | 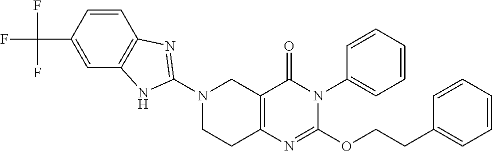 | 532.1 |
| 49 | 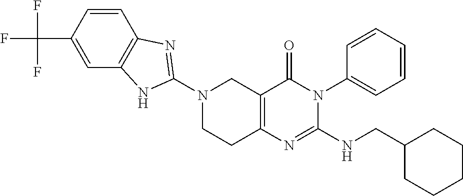 | 523.2 |
| 50 | 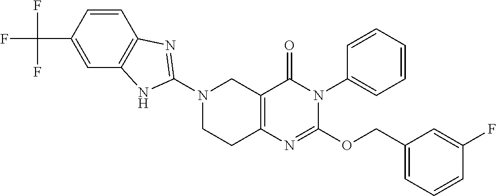 | 536.1 |
| 51 | 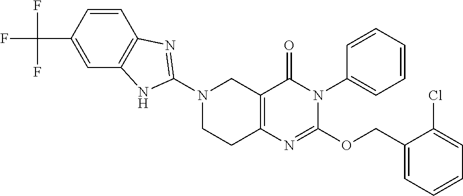 | 552.1 |
| 52 | 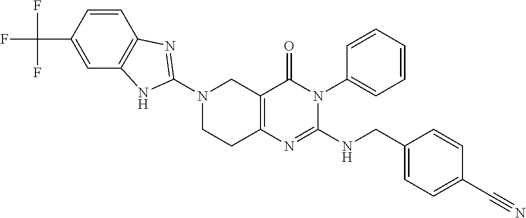 | 542.2 |

TABLE 13

| | | |
|---|---|---|
| 53 | 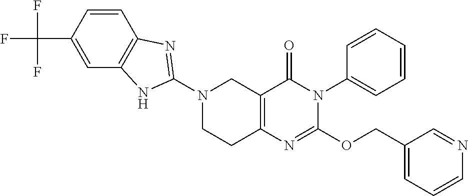 | 519.2 |
| 54 | 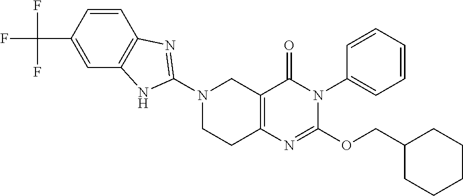 | 524.1 |
| 55 | 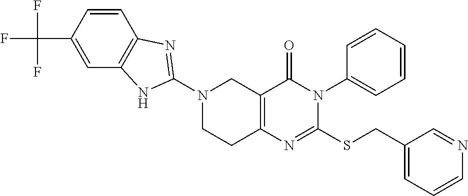 | 535.2 |
| 56 | 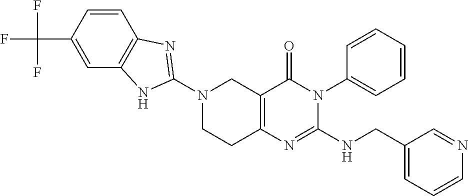 | 518.2 |
| 57 | 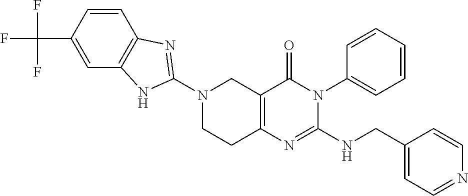 | 518.2 |
| 58 | 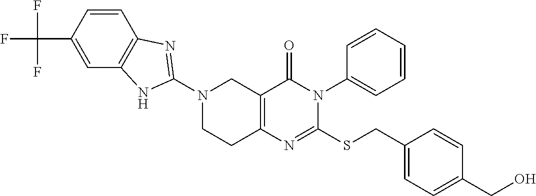 | 564.1 |

TABLE 14

| | | |
|---|---|---|
| 59 | 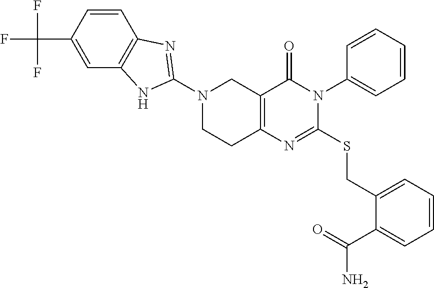 | 577.2 |
| 60 | 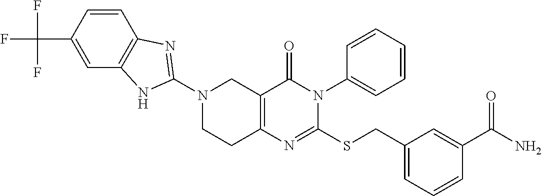 | 577.2 |
| 61 | 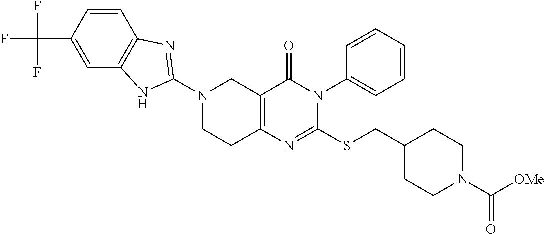 | 599.2 |
| 62 | 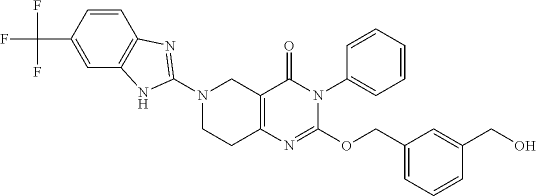 | 548.1 |
| 63 | 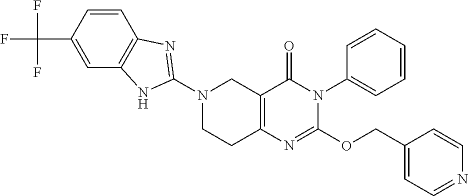 | 519.2 |
| 64 | 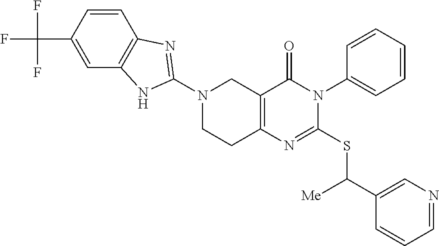 | 549.1 |

TABLE 15

| | | |
|---|---|---|
| 65 | 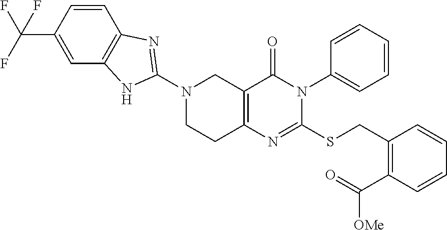 | 592.2 |
| 66 | 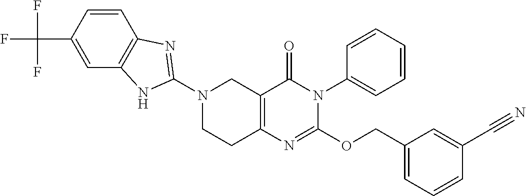 | 543.1 |
| 67 | 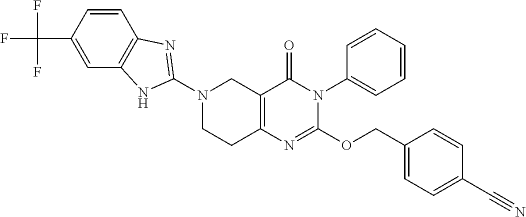 | 543.2 |
| 68 | 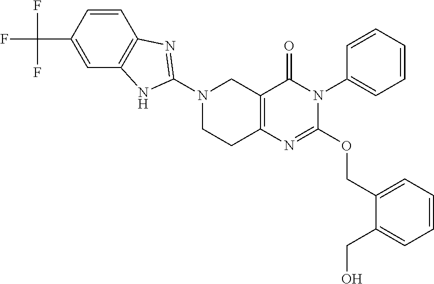 | 548.1 |
| 69 | 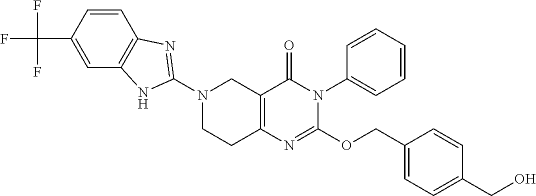 | 548.1 |
| 70 | 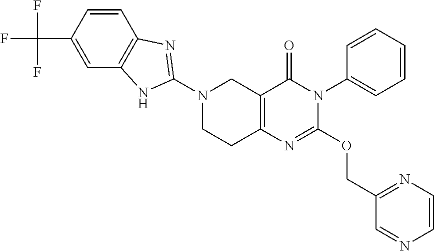 | 520.2 |

TABLE 16

| | | |
|---|---|---|
| 71 | 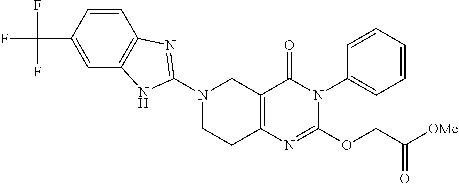 | 500.1 |
| 72 | 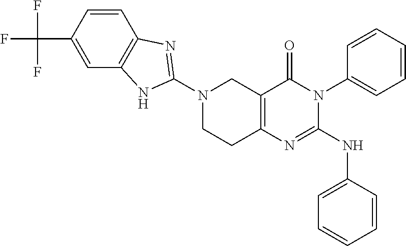 | 503.2 |
| 73 | 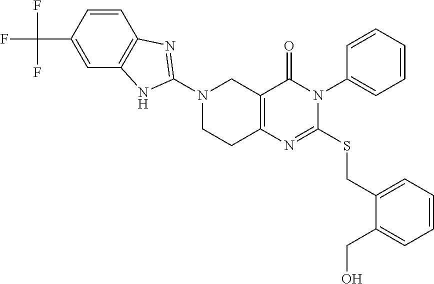 | 564.2 |
| 74 | 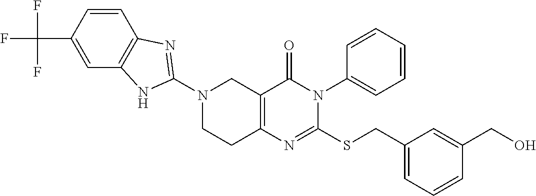 | 564.2 |
| 75 | 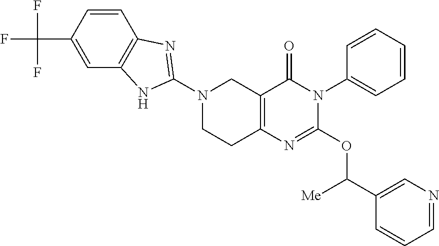 | 533.2 |
| 76 | 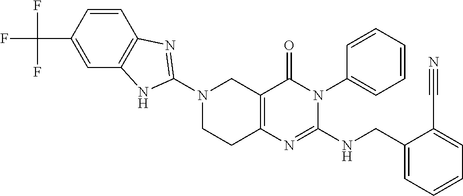 | 542.2 |

TABLE 17

| | | |
|---|---|---|
| 77 | 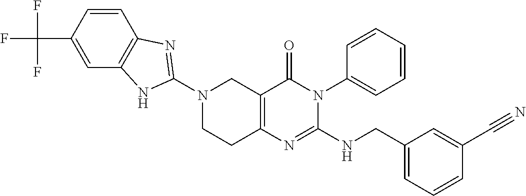 | 542.2 |
| 78 | 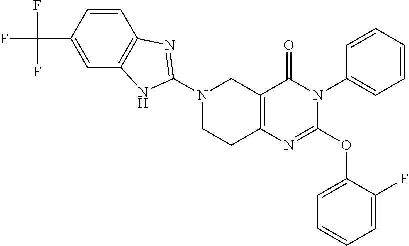 | 522.1 |
| 79 | 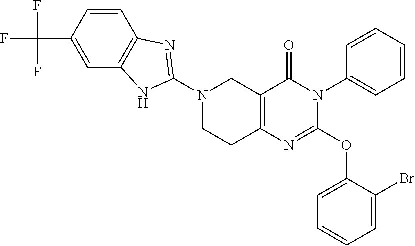 | 582.1/ 584.1 |
| 80 | 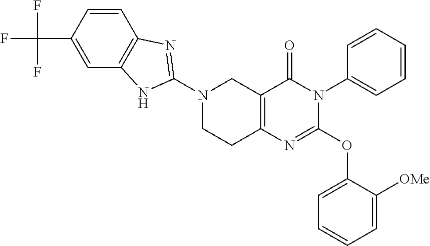 | 534.2 |
| 81 | 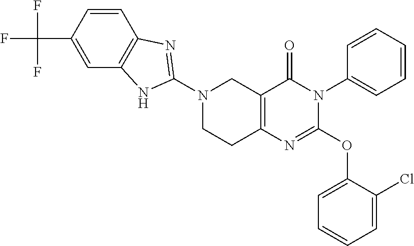 | 538.2/ 540.2 |
| 82 | 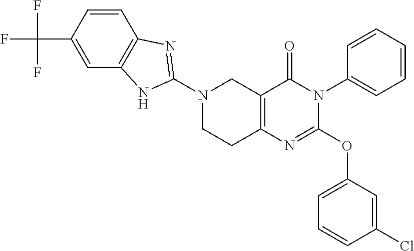 | 538.2 |

TABLE 18

| | | |
|---|---|---|
| 83 | 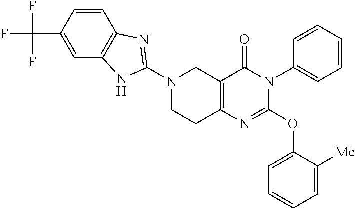 | 518.2 |
| 84 | 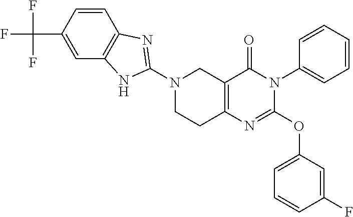 | 522.1 |
| 85 | 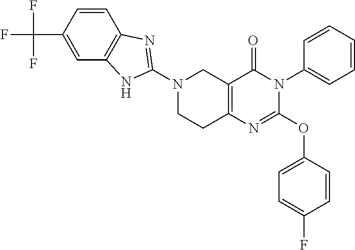 | 522.1 |
| 86 | 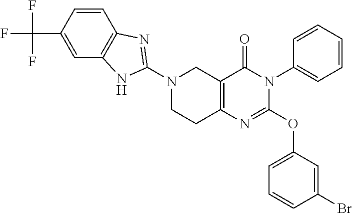 | 582.1/ 584.0 |
| 87 | 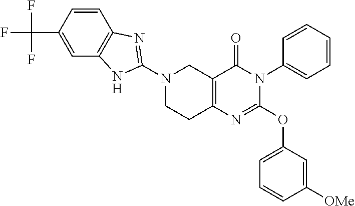 | 534.1 |

TABLE 19

| | | |
|---|---|---|
| 88 | 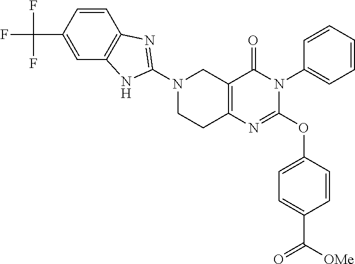 | 562.1 |
| 89 | 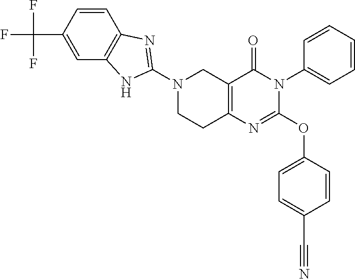 | 529.1 |
| 90 | 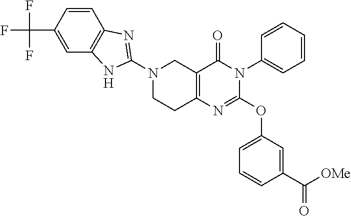 | 562.2 |

In addition, as Reference Examples, processes for producing compounds used for producing a compound according the present invention are described below.

### Reference Example 1

### Production of tert-butyl-4-oxo-3-phenyl-2-benzylthio-3,5,7,8-tetrahydropyrido[4,3-d]pyrimidin-6(4H)-carboxylate

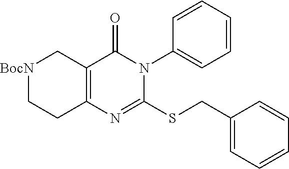

To a solution of tert-butyl-4-oxo-3-phenyl-2-thioxo-1,3,4,5,7,8-hexahydropyrido[4,3-d]pyrimidin-6(2H)-carboxylate in N,N-dimethylformamide, 0.5 ml of 1,8-diazabicyclo[5.4.0]-7-undecene was added under ice-cooling, followed by addition of 0.3 ml of benzylbromide and the reaction mixture was stirred overnight at temperature reset to room temperature.

Water was added to the reaction solution, and the mixture was extracted with ethyl acetate. The organic layer was washed with a saturated saline solution, dried over anhydrous sodium sulfate, and concentrated under reduced pressure. The resultant residue was purified by silica gel chromatography to yield the title compound.

¹H-NMR (DMSO-D₆) δ: 1.44 (s, 9H), 2.70 (t, J=5.6 Hz, 2H), 3.62 (t, J=5.6 Hz, 2H), 4.18 (s, 2H), 4.31 (s, 2H), 7.21-7.35 (m, 7H), 7.49-7.56 (m, 3H)

ESI-MS Found: m/z 450 [M+H]+

### Reference Example 2

### Production of 2-benzylthio-3-phenyl-3,5,7,8-tetrahydropyrido[4,3-d]pyrimidin-4(3H)-one hydrochloride

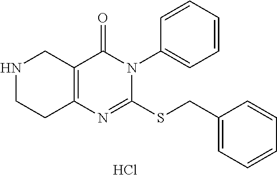

To a solution of tert-butyl-4-oxo-3-phenyl-2-benzylthio-3,5,7,8-tetrahydropyrido[4,3-d]pyrimidin-6(4H)-carboxylate obtained in Reference Example 1 in dioxane, 4M hydrochloric acid-dioxane was added at room temperature. After one hour, the solvent was distilled off under reduced pressure to yield the title compound.

ESI-MS Found: m/z 350 [M+H]+

### Reference Example 3

### Production of tert-butyl-4-oxo-3-phenyl-2-((pyridin-3-ylmethyl)thio)-3,5,7,8-tetrahydropyrido[4,3-d]pyrimidin-6(4H)-carboxylate

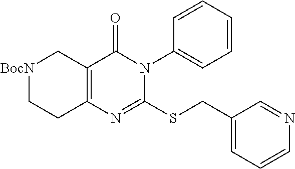

Using tert-butyl-4-oxo-3-phenyl-2-thioxo-1,3,4,5,7,8-hexahydropyrido[4,3-d]pyrimidin-6(2H)-carboxylate and 3-pyridylmethylbromide hydrobromate, the title compound was obtained by the same method as in Reference Example 1.

¹H-NMR (CDCl₃) δ: 1.61 (s, 9H), 2.76 (t, J=5.6 Hz, 2H), 3.71 (t, J=5.6 Hz, 2H), 4.25 (s, 2H), 4.34 (s, 2H), 7.20-7.29 (m, 4H), 7.51-7.66 (m, 3H), 8.47-8.49 (m, 1H), 8.60 (d, J=1.6 Hz, 3H)

ESI-MS Found: m/z 451 [M+H]+

### Reference Example 4

### Production of 2-((pyridin-3-ylmethyl)thio)-3-phenyl-3,5,7,8-tetrahydropyrido[4,3-d]pyrimidin-4(3H)-one dihydrochloride

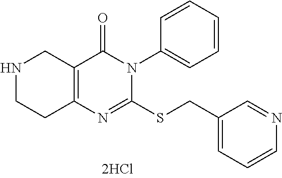

Using tert-butyl-4-oxo-3-phenyl-2-((pyridin-3-ylmethyl)thio)-3,5,7,8-tetrahydropyrido[4,3-d]pyrimidin-6(4H)-carboxylate obtained in Reference Example 3, the title compound was obtained by the same method as in Reference Example 2.

ESI-MS Found: m/z 351 [M+H]+

### Reference Example 5

### Production of tert-butyl-4-oxo-3-phenyl-2-((pyridazin-4-ylmethyl)thio)-3,5,7,8-tetrahydropyrido[4,3-d]pyrimidin-6(4H)-carboxylate

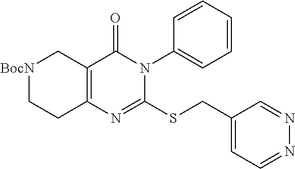

Using tert-butyl-4-oxo-3-phenyl-2-thioxo-1,3,4,5,7,8-hexahydropyrido[4,3-d]pyrimidin-6(2H)-carboxylate and 4-pyridazinemethyl chloride hydrochloride, the title compound was obtained by the same method as in Reference Example 1.

¹H-NMR (CDCl3) δ: 1.56 (s, 9H), 2.71 (t, J=6.0 Hz, 2H), 3.68 (t, J=6.0 Hz, 2H), 4.20 (s, 2H), 4.33 (s, 2H), 7.21-7.24 (m, 2H), 7.44-7.55 (m, 4H), 9.11 (d, J=5.6 Hz, 1H), 9.22 (s, 1H)

ESI-MS Found: m/z 452 [M+H]+

### Reference Example 6

### Production of 2-((pyridazin-4-ylmethyl)thio)-3-phenyl-3,5,7,8-tetrahydropyrido[4,3-d]pyrimidin-4(3H)-one dihydrochloride

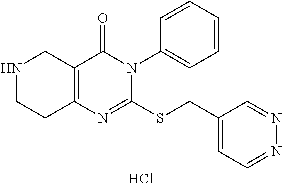

Using tert-butyl-4-oxo-3-phenyl-2-((pyridazin-4-ylmethyl)thio)-3,5,7,8-tetrahydropyrido[4,3-d]pyrimidin-6(4H)-carboxylate obtained in Reference Example 3, the title compound was obtained by the same method as in Reference Example 2.

ESI-MS Found: m/z 352 [M+H]+

The usefulness of the compound represented by the formula (I) as a medicament is proved, for example, in the test described below.

### Cloning of Human MGAT2 Gene and Expression in Yeast

Human MGAT2 genes were amplified by PCR using primers described below from human cDNA library (Clontech).

| | |
|---|---|
| MGAT1F: | 5′-TTGAATTCATAATGGTAGAGTTCGCGCCCTTGT-3′ |
| MGAT2R: | 5′-ACCGGTGCAGAACTCCAAGTGCTGGT-3′ |

The amplified human MGAT2 genes were introduced into a yeast expression vector pPICZA (Invitrogen). The resultant expression plasmid was introduced into an yeast (Pichia pastris) by electroporation to produce a recombinant yeast. The recombinant yeast was cultured in the presence of 0.5% methanol for 72 hours, and the cells were crushed using glass beads in 10 mM Tris pH 7.5, 250 mM sucrose and 1 mM EDTA, followed by adjusting the membrane fraction by centrifugation to use the adjusted membrane fraction as an enzyme source.

### MGAT2 Inhibitory Activity Test

To the reaction liquid having the following composition: 100 mM Tris pH 7.0, 5 mM MgCl₂, 200 mM sucrose, 100 μM monoolein, 500 μM phosphatidylcholine, 15 μM [¹⁴C]-oleoyl-CoA, 0.1 pg of test substance, MGAT2-expressed yeast membrane fraction, was added, and the mixture having a volume of 100 μl was incubated at 37° C. for 30 minutes. To the reaction solution, 100 μl of 2-propanol/heptan (80:20) was added, the mixture was stirred well, followed by addition of 200 μl of heptane and further stirring the mixture. After centrifugation, the heptane layer was collected, 80 μl of ethanol/0.1N NaOH/H₂O (50:5:45) was added, the mixture was thus stirred, followed by recentrifuging the mixture and collecting the heptane layer. After exsiccation of the resultant heptane layer, 100 μl of Microscint 0 (PerkinElmer) was added, and the radioactivity was measured with a liquid scintillation counter. The inhibitory activity was calculated from the following formula:

Inhibition rate=100−(radioactivity in case of addition of test compound−background)/(radioactivity in case of addition of no test compound−background)×100

wherein the background means the radioactivity in case of addition of no membrane fraction.

The MGAT2 inhibitory activity of the compound according to the present invention by the aforementioned method is shown below.

TABLE 20

| Example | MGAT2 Inhibitory Activity |
|---|---|
| Number | IC₅₀ (nM) |
| 1 | 817 |
| 2 | 184 |
| 3 | 56 |
| 4 | 114 |

The aforementioned results exhibit that the compound (I) according to the present invention has strong MGAT2 inhibitory activity.

1. A compound of a formula I: 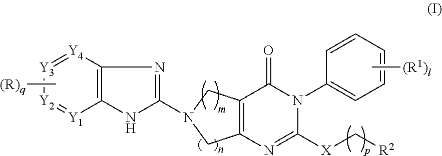
or a pharmaceutically acceptable salt thereof, wherein:
R¹ is each independently selected from the group consisting of:
lower alkyl group unsubstituted or substituted with 1 to 3 halogen atoms,
lower alkoxy group unsubstituted of substituted with 1 to 3 halogen atoms, and halogen atoms;
R² is selected from the group consisting of:
phenyl group,
5- or 6-membered heteroaryl group containing 1 to 3 hetero atom(s) selected from the group consisting of nitrogen atom, sulfur atom and oxygen atom,
C₃₋₇ cycloalkyl group, wherein one carbon atom of the C₃₋₇ cycloalkyl group may be replaced with nitrogen atom, and
cyano or lower alkoxycarbonyl group;
said phenyl group, 5- or 6-membered heteroaryl group containing 1 to 3 hetero atom(s) selected from the group consisting of nitrogen atom, sulfur atom and oxygen atom, and C₃₋₇ cycloalkyl group wherein one carbon atom of the C₃₋₇ cycloalkyl group may be replaced with nitrogen atom, may be substituted with the same or different, 1 to 3 groups selected from the groups consisting of:
lower alkyl group unsubstituted or substituted with the same or different, 1 to 3 halogen atom(s) or hydroxy group,
lower alkoxy group unsubstituted or substituted with the same or different, 1 to 3 halogen atom(s),
lower alkoxy carbonyl group,
cyano group,
carbamoyl group,
mono- or di-lower alkyl carbamoyl group,
hydroxy group, and
halogen atom;
R is each independently halogen atom, lower alkyl group or lower alkoxy group;
said lower alkyl group and lower alkoxy group may be substituted with the same or different, 1 to 3 halogen atom(s);
X is selected from the group consisting of NR′, oxygen atom and sulfur atom;
R′ is lower alkyl group;
each of Y₁, Y₂, Y₃ and Y₄ is all CH, or 1 or 2 of them is nitrogen atom, and the rest are CH;
l is an integer of 0 to 3;
m and n is an integer of 1 or 2;
p is an integer of 0 to 2; and
q is an integer of 1 to 3.

2. The compound according to claim 1, wherein: each of Y₁, Y₂, Y₃ and Y₄ is all CH; or the pharmaceutically acceptable salt thereof.

3. The compound according to claim 2, wherein: X is an oxygen atom or sulfur atom; or the pharmaceutically acceptable salt thereof.

4. The compound according to claim 3, wherein: l is 0; or the pharmaceutically acceptable salt thereof.

5. The compound according to claim 4, wherein: m and n is each different, 1 or 2; or the pharmaceutically acceptable salt thereof.

6. The compound according to claim 1, wherein: R is trifluoromethyl group or fluorine atom; or the pharmaceutically acceptable salt thereof.

7. The compound according to claim 3 wherein: p is 0 or 1; or the pharmaceutically acceptable salt thereof.

8. The compound according to claim 1, wherein the compound represented by the formula I is:
6-(5,6-difluoro-1H-benzimidazol-2-yl)-3-pheny-2-[(phenylmethyl)thio]-5,6,7,8-tetrahydropyrido[4,3-d]pyrimidin-4(3H)-one,
6-(5-trifluoromethyl-1H-benzimidazol-2-yl)-3-phenyl-2-[(phenylmethyl)thio]-5,6,7,8-tetrahydropyrido[4,3-d]pyrimidin-4(3H)-one,
3-phenyl-2-[(pyridin-3-ylmethyl)thio]-645-(trifluoromethyl)-1H-benzimidazol-2-yl]-5,6,7,8-tetrahydropyrido[4,3-d]pyrimidin-4(3H)-one, or
3-phenyl-2-[(pyridazin-4-ylmethyl)thio]-6-[5-(trifluoromethyl)-1H-benzimidazol-2-yl]-5,6,7,8-tetrahydropyrido[4,3-d]pyrimidin-4(3H)one,
or a pharmaceutically acceptable salt thereof.

9. (canceled)

10. (canceled)

11. A pharmaceutical composition comprising the compound of claim 1 and a pharmaceutically acceptable carrier.

12. A method of treating hyperlipidemia, diabetes or obesity comprising administering to a patient in need thereof a therapeutically effective amount of a compound of claim 1.
